(19) United States
(12) Patent Application Publication
(10) Pub. No.: US 2008/0077021 A1
(43) Pub. Date: Mar. 27, 2008

(54) Blood Pressure Monitor Calibration Device And Method For Calibrating A Blood Pressure Monitor

(75) Inventors: Roman S. Ferber, West Bloomfield, MI (US); Hung Ching-Hsi, Taipei (TW)

Correspondence Address: BROOKS KUSHMAN P.C., 1000 TOWN CENTER, TWENTY-SECOND FLOOR, SOUTHFIELD, MI, 48075, US

(73) Assignee: FKA DISTRIBUTING CO. D/B/A HOMEDICS, INC., Commerce Township, MI (US)

(21) Appl. No.: 11/535,689

(22) Filed: Sep. 27, 2006

(51) Int. Cl. A61B 5/02

(52) U.S. Cl. 600/485

(57) ABSTRACT

According to one embodiment of the present invention, a method for calibrating a blood pressure monitor is disclosed. The method includes generating a first training pressure point signal corresponding to a first pressure value; transmitting the first training pressure point signal to a pressure sensor of a blood pressure monitor (BPM); receiving a first sensed pressure value sensed by the pressure sensor; generating a second training pressure point signal corresponding to a second pressure value; transmitting the second training pressure point signal to the pressure sensor of the BPM; receiving a second sensed pressure value sensed by the pressure sensor; receiving BMP calibration data; and generating new calibration data based on the first pressure value, the second pressure value, the first sensed pressure value, the second sensed pressure value and the BMP calibration data.

### BACKGROUND OF THE INVENTION

1. Field of the Invention

One aspect of the present invention relates to a blood pressure monitor calibration device. Another aspect of the present invention relates to a calibration device for a blood pressure monitor and a method of using the same.

2. Background Art

Many diseases, such as blood vessel diseases, may result from improper diet and/or lack of exercise. A healthy lifestyle, e.g. one that includes regular exercise and proper dieting, may lessen the risk of developing such diseases. An individual's blood pressure can be compared with a range of healthy blood pressures to determine whether the individual is engaging in healthy living. Monitoring an individual's blood pressure over time can be instrumental in maintaining a healthy living regiment. For instance, if an individual's blood pressure exceeds the healthy range, the individual's level of exercise can be increased and/or the individual's diet can be changed in an effort to positively adjust the individual's blood pressure into a healthy range.

Many devices exist for measuring blood pressure. One non-limiting example of a blood pressure measuring device is a manometer, which typically includes a pressure gauge for measuring blood pressure. Individuals may have difficulty operating conventional manometers due to their relatively bulky size and complexity of operation. In many instances, individuals must travel to a hospital so that a medical professional, e.g. a nurse, can operate the manometer for the individual, which can present an inconvenience.

At least one proposed manometer attempts to address these problems by providing a design with relatively good portability and a facile user interface. This design includes functionality for measuring an individual's blood pressure and storing and displaying one or more measurements for blood pressure trending analysis. The measurement and trend data provides a relatively solid reference for medical care professionals.

However, such manometers typically require calibration to deliver precise blood pressure data. Calibration may be warranted at many points during the life of the manometer, e.g. during manufacturing and by medical care professionals and/or users after substantial use. The calibration process may include removing the outer casing of the manometer to obtain access to and calibrate the internal components thereof. While this step can be avoided at the manufacturing facility by calibrating before the outer casing is secured, this is not the case after the manometer is put into use. The removal step can be cumbersome for users and/or medical care professionals.

In light of the foregoing, there is a need for a blood pressure monitor calibration device that addresses one or more of the disadvantages identified above. What is also needed is a method for calibrating a blood pressure monitor that addresses one or more of the disadvantages identified above.

### SUMMARY OF THE INVENTION

According to one embodiment of the present invention, a method for calibrating a blood pressure monitor is disclosed. The method includes generating a first training pressure point signal corresponding to a first pressure value; transmitting the first training pressure point signal to a pressure sensor of a blood pressure monitor (BPM); receiving a first sensed pressure value sensed by the pressure sensor; generating a second training pressure point signal corresponding to a second pressure value; transmitting the second training pressure point signal to the pressure sensor of the BPM; receiving a second sensed pressure value sensed by the pressure sensor; receiving BMP calibration data; and generating new calibration data based on the first pressure value, the second pressure value, the first sensed pressure value, the second sensed pressure value and the BMP calibration data.

According to another embodiment of the present invention, a calibration device for a blood pressure monitor is disclosed. The calibration device includes a central processing unit (CPU) for executing machine instructions; a memory for storing machine instructions that are to be executed by the CPU; and a communication interface for electrically communicating with a blood pressure monitor. The machine instructions when executed by the CPU implement the following functions: receiving a first training pressure point signal corresponding to a first pressure value; transmitting the first training pressure point signal to a pressure sensor of a blood pressure monitor (BPM); receiving a first sensed pressure value sensed by the pressure sensor; generating a second training pressure point signal corresponding to a second pressure value; transmitting the second training pressure point signal to the pressure sensor of the BPM; receiving a second sensed pressure value sensed by the pressure sensor; receiving BMP calibration data; and generating new calibration data based on the first pressure value, the second pressure value, the first sensed pressure value, the second sensed pressure value and the BMP calibration data.

According to yet another embodiment of the present invention, a calibration device for a blood pressure monitor is disclosed. The device includes a central processing unit (CPU) for executing machine instructions; a memory for storing machine instructions that are to be executed by the CPU; a communication interface for electrically communicating with a blood pressure monitor; and a pressure sensor for sensing a pressure value corresponding to a pressure point signal. The machine instructions when executed by the CPU implement the following functions: receiving a pressure point signal (PS) corresponding to a pressure value (PV); transmitting the PS to the pressure sensor and the communication interface; receiving a sensed pressure value (SP_{B}) from the blood pressure monitor through the communication interface; receiving a sensed pressure value (SP_{C}) from the pressure sensor; comparing the difference between SP_{B} and SP_{C} with a tolerable range to obtain a calibration status of either “yes” if the difference is outside of the tolerable range and “no” if the difference is within the tolerable range; and transmitting the calibration status to determine whether to initiate a calibration sequence.

These and other aspects of the present invention will be better understood in view of the following detailed description of the invention.

### BRIEF DESCRIPTION OF DRAWINGS

The features of the invention believed to be novel are set forth with particularity in the appended claims. The invention itself however may be best understood by reference to the following detailed description of the invention, which describes certain exemplary embodiments of the invention, taken in conjunction with the accompanying drawings in which:

FIG. 1 shows a block diagram of a blood pressure monitor and calibration device according to one embodiment of the present invention;

FIG. 2 depicts an operational flowchart of the calibration device of FIG. 1 according to one embodiment of the present invention;

FIG. 3 depicts an operational flowchart of a calibration method for a blood pressure monitor according to one embodiment of the present invention; and

FIG. 4 depicts an operational flowchart of a training method for a blood pressure monitor according to one embodiment of the present invention.

### DETAILED DESCRIPTION OF EMBODIMENTS OF THE PRESENT INVENTION

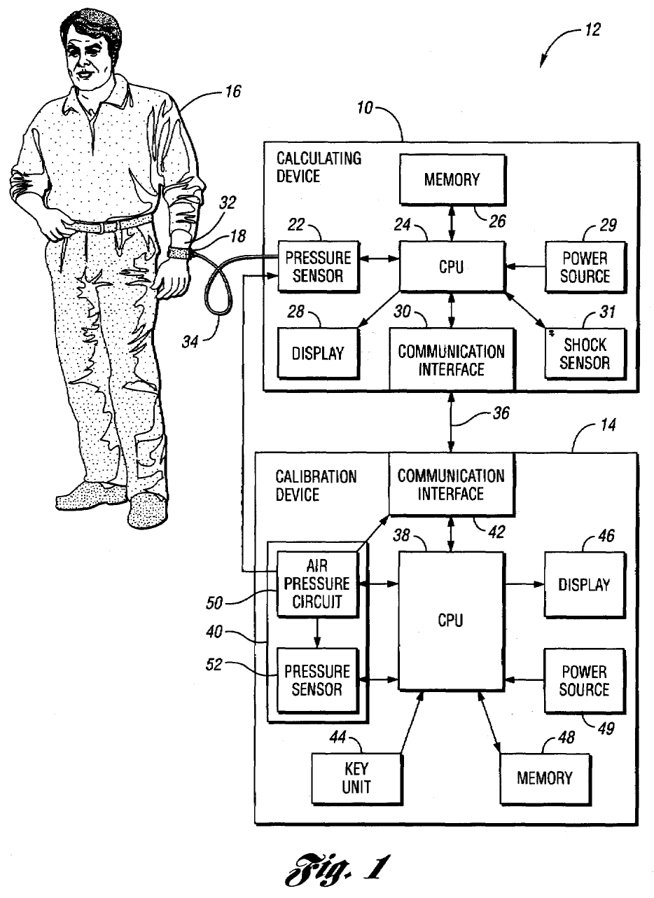

FIG. 1 shows a calculating device 10 of a blood pressure monitor (BPM) 12 and a calibration device 14 according to one embodiment of the present invention. As depicted in FIG. 1, the BPM is a wrist-type BPM, although other types of BPMs can be used in accordance with this invention, for example, upper arm BPMs. The BPM can utilize an oscillometric method known to one of ordinary skill in the art to measure the blood pressure of a patient 16. A non-limiting example of a BPM that utilizes such a method is an electronic manometer.

The BPM 12 includes a cuff 18, a pump (not shown) for delivering pressure to the cuff, and the calculating device 10, which includes a pressure sensor 22, a central processing unit (CPU) 24, a memory 26, a display 28, a power source 29 and a communication interface 30. In at least one embodiment, the cuff 18, the pump and the calculating device 10 can be contained within a single unit, i.e. a single housing. The cuff 18 at least partially surrounds a wrist 32 of the patient 16. The pressure sensor 22 is electrically connected to the cuff 18 through a wire 34. It should be appreciated that in other embodiments, a wireless connection can be used for communication between the cuff 18 and pressure sensor 22. The pressure sensor 22 measures the blood pressure within the cuff 18 and transmits a sensed blood pressure value to CPU 24. The CPU 24 calculates blood pressure values, such as the systolic blood pressure and the diastolic blood pressure, based on the sensed blood pressure and data contained in memory 26, which can include data for calculating blood pressure values and data for calibrating the sensed blood pressure measurements. The display 28 displays blood pressure values calculated by CPU 24. In at least one embodiment, the memory 26 can be configured to store machine instructions, e.g. software and/or firmware and CPU 24 can be adapted to execute the machine instructions. The machine instructions can include a counting routine for counting the number of uses of the BPM 12. The memory can store a recalibration value defined as a number of operations since last calibration. The value can be in the range of 2,000 or 5,000 to 7,000 to 15,000. In at least one embodiment, the value is 10,000. The counting routine can also include functionality for triggering display of a recalibration alert on display 28 upon reading the recalibration value.

In at least one embodiment, the BPM 12 can include a shock sensor 31 for sensing whether the BPM 12 has undergone a shock event, e.g. whether it has been dropped or exposed to extreme temperatures (e.g. T≧120° C.). Upon sensing a shock event, a shock signal can be sent to CPU 24, which can be configured to execute machine instructions for causing display of a shock alert on display 28.

The calculating device 10 is adapted to communicate with other devices, such as calibration device 14, through communication interface 30, which can be configured to receive and transmit data from a wired and/or wireless connection depending on the implementation of the present invention. According to FIG. 1, the communication interface 30 is suitable for receiving and transmitting data through wired connection 36.

The power source 29 is electrically connected to the CPU 24 and provides power to pressure sensor 22, memory 26, display 28, and communication interface 30.

The calibration device 14 includes a central processing unit (CPU) 38, a pressure unit 40, a communication interface 42, a key unit 44, a display 46, memory 48, and power source 49. The calibration device 14 is electrically connected to the calculating device 10 through communication interface 42 and wired connection 36, although a wireless connection is also contemplated. The communication interface 42 can include an RS-232 interface.

The CPU 38 can be adapted to carry out one or more steps of a calibration process for calibrating BPM 12. In at least one embodiment, the CPU 38 is adapted to execute machine instructions for carrying out one or more steps of the calibration process and memory 48 is adapted to store machine instructions that are to be executed by the CPU 38. The memory 48 can be non-volatile memory, for example, read-only memory or flash memory, and can be configured to store pressure values for calibration and/or calibration software. Moreover, memory 48 can be configured as calibration firmware.

The pressure unit 40 is electrically connected to the CPU 38 and the BPM 12. The pressure unit 40 includes an air pressure circuit 50 for transmitting pressure point signals to the CPU 38 and the pressure sensor 22 of the BPM 12. In at least one embodiment, the transmission of the pressure point signal from air pressure circuit 50 to pressure sensor 22 passes through communication interface 42, although in other embodiments, the transmission can occur directly between air pressure circuit 50 and pressure sensor 22. The air pressure circuit 50 can also transmit pressure point signals to a pressure sensor 52, which generates a sensed pressure value based on each pressure point signal. The generated sensed pressure value can be transmitted to CPU 38 by pressure sensor 52.

In at least one embodiment, the pressure unit 40 additionally includes a motor, an electronic valve, a tank and an air conduit (e.g. a tube) to generate and deliver pressurized air at various pressures to the pressure sensor 22 and/or pressure sensor 52. The pressure point signals can be generated and delivered as the pressurized air at desired pressures.

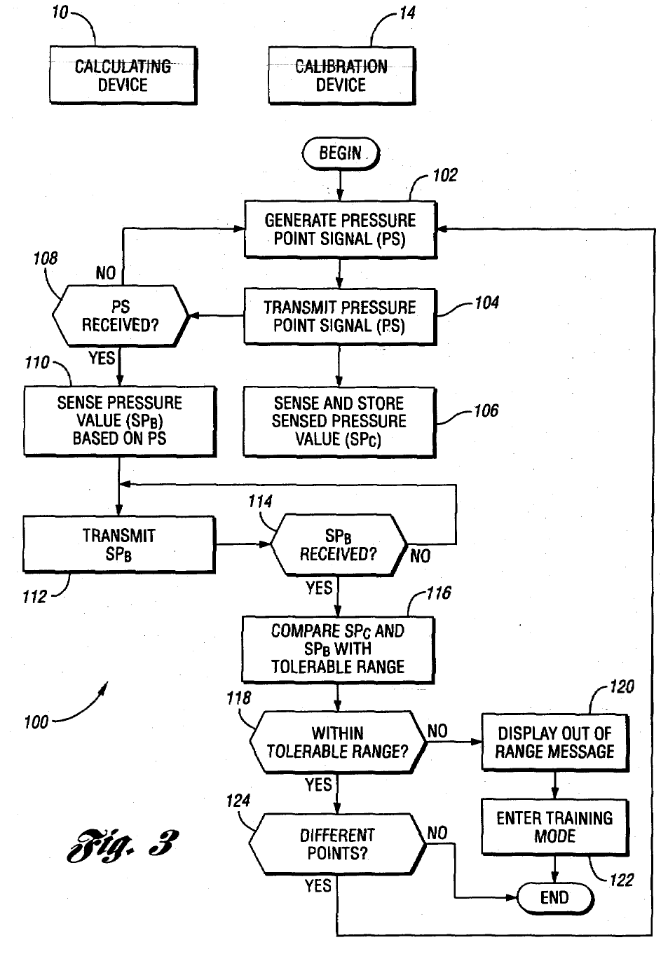

The key unit 44 is electrically connected to the CPU 38. The key unit 44 can include one or more input buttons for selecting one or more operation modes of the calibration device 14. Non-limiting examples of operation modes include a calibration mode for calibrating the BPM 12 and a training mode for training the BPM 12. The key unit 44 can also include a test button (not shown). Upon activation, e.g. pressing the test button, the calibrating device 14 can execute one or more steps for identifying whether a calibration condition has been met, as described in FIG. 3.

The display 46 is electrically connected to the CPU 38. The display can be configured to display one or more calibration values processed by the CPU 38. A non-limiting example of display 46 is a liquid crystal display (LCD).

The power source 49 is electrically connected to the CPU 38 and provides power to pressure unit 40, communication interface 42, key unit 44, and display 46.

As depicted in FIG. 1, calculating device 10 and calibration device 14 are two separate devices that are interconnected through communication interfaces 36 and 42. In other embodiments of the present invention, it should be appreciated that the calculating device 10 and calibration device 14 can be contained within a single unit. In such embodiments, parts can be omitted, added and/or rearranged to obtain the single unit. For example, by combining the calculating device 10 and calibration device 14, the extra CPU, power source, memory and display can be omitted, although two pressure sensors can be maintained, i.e. one for measuring blood pressure and one for calibrating the measurements.

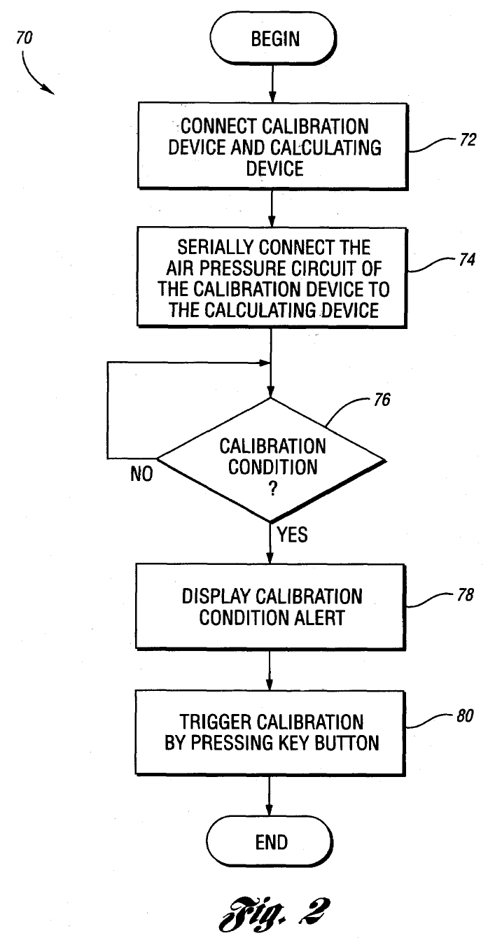

FIG. 2 depicts an operational flowchart 70 for triggering a calibration process according to one embodiment of the present invention. In block 72 of flowchart 70, the communication interface 42 of the calibration device 14 is connected to the calculating device 10. According to block 74, the air pressure circuit 50 of the pressure unit 40 is serially connected to the calculating device 10.

Decision block 76 tests whether or not a calibration condition has been met for the BPM 12. In at least one embodiment, this determination is made by reference to the process set forth in FIG. 3, which is described in detail below. In at least one embodiment, the calibration condition can be met if the number of operations value since last calibration is met. If the value obtained from decision block 76 is no, then the process loops back to decision block 76, and the calibration condition is re-tested after a period of time, e.g. one to twelve months. If the value obtained from decision block 76 is yes, then an alert can be displayed on display 46 for alerting the user that the calibration condition has been met as shown in block 78. In block 80, a key button on the key unit 44 can be pressed to trigger a calibration process. In at least one embodiment, the calculating device 10 enters a calibration mode for awaiting one or more pressure point signals from the air pressure circuit 50 of the calibration device 14.

FIG. 3 depicts an operation flowchart 100 of a calibration method according to one embodiment of the present invention. According to block 102, the calibration device 14 generates one or more pressure point signals for comparison purposes. In at least one embodiment, this step occurs while the calculating device 10 is connected to the calibration device 14, and the pressure point signal can correspond to a pressure in the range of 200 or 250 to 300 or 350 mm Hg.

In at least one embodiment, the calibration device 14 can be configured to enter calibration mode and perform a calibration sequence prior to each operation of BPM 12.

In block 104, the air pressure circuit 50 transmits the pressure point signal (PS) to the pressure sensor 52 of the calibration device 14 and the pressure sensor 22 of the calculating device 10.

In block 106, the pressure sensor 52 of the calibration device 14 senses the pressure of the pressure point signal to obtain a sensed pressure value (SP_{C}), which is stored in memory 48 of the calibration device 14.

In decision block 108, the calculating device 10 tests whether the pressure point signal has been received. If no, the process is returned to block 102 for generating a new pressure point signal. If yes, the pressure sensor 22 of BPM 12 senses the pressure of the pressure point signal to obtain a sensed pressure value (SP_{B}), as depicted in block 110.

According to block 112, SP_{B} is transmitted to the calibration device 14. In decision block 114, the calibration device 14 tests whether SP_{B} has been received from the calculating device 10. If no, the process is returned to block 112 for re-transmitting the SP_{B}. If yes, then SP_{B} and SP_{C} are compared at block 116 to determine whether the pressure values are within a tolerable range of each other. In at least one embodiment, the tolerable range can be in the range of 0 mm Hg to 50 mm Hg, 40 mm Hg, 30 mm Hg, 20 mm Hg, 10 mm Hg, 5 mm Hg, 3 mm Hg, or 1 mm Hg. Alternatively, the tolerable range for the SP_{B} can be defined as a percentage plus and minus of SP_{C}. The percentage can be in the range of 1% to 5%, 10%, 20%, or 25%. For instance, if SP_{C} is 250 mm Hg and the percentage is 10%, then the percentage plus and minus values are 275 mm Hg and 225 mm Hg, i.e. the defined tolerable range for the SP_{B}.

In decision block 118, the calibration device 14 tests whether the SP_{B} and SP_{C} are within the tolerable range. If no, then an “out of range” message can be displayed on display 24 (block 120) and the calibration device enters training mode (block 122). If the sensed pressure is within the tolerable range, then the process can be repeated for different pressure points, as depicted in decision block 124. For example, the process can be carried out for 0, 50, 150 and/or 250 mm Hg as different pressure points.

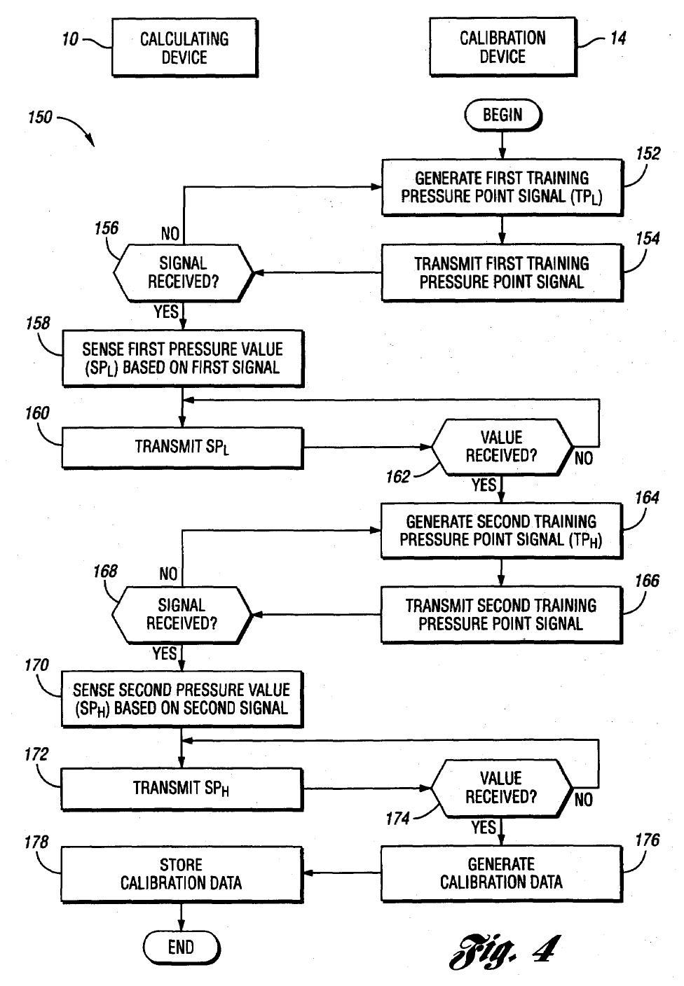

FIG. 4 depicts an operational flowchart 150 of a training method according to one embodiment of the present invention.

In block 152, the calibration device 14 generates a first training pressure point signal for comparison purposes. In at least one embodiment, the pressure point signal corresponds to a relatively low level of pressure (TP_{L}), which can be in the range of 200 or 250 to 250 or 300 mm Hg.

In block 154, the calibration device 14 transmits the first training pressure point signal to the pressure sensor 22 of the calculating device 10. In decision block 156, the calculating device 10 tests whether the first training pressure point signal has been received. If no, the process is returned to block 152 for re-generating the first training pressure point signal. If yes, the pressure sensor 22 senses the pressure of the pressure point signal to obtain a sensed first pressure value (SP_{L}), as depicted in block 158.

According to block 160, SP_{L} is transmitted to the calibration device 14. In decision block 162, the calibration device 14 tests whether SP_{L} has been received from the calculating device 10. If no, the process is returned to block 160 for re-transmitting SP_{L}. If yes, the calibration device 14 generates a second training pressure point signal for comparison purposes, as depicted in block 164. In at least one embodiment, the pressure point signal corresponds to a relatively high level of pressure (TP_{H}), which can be in the range of 250 or 300 to 300 to 350 mm Hg.

In block 166, the calibration device 14 transmits the second training pressure point signal generated in block 164 to the calculating device 10 of BPM 12. In decision block 168, the calculating device 10 tests whether SP_{H} has been received. If no, the process is returned to block 164 for re-generating the second training pressure point signal. If yes, the pressure sensor 22 senses the pressure of the second pressure point signal to obtain a second sensed pressure value (SP_{H}), as depicted in block 170.

According to block 172, SP_{H} is transmitted to the calibration device 14. In decision block 174, the calibration device 14 tests whether SP_{H} has been received by the calibration device 14. If no, the process is returned to block 172 for re-transmitting SP_{H}. If yes, then the calibration device 14 generates calibration data, as depicted in block 176. The calibration data can include, but is not limited to, TP_{L} and TP_{H}.

In block 178, the calibration data and SP_{L} and SP_{H} are stored in the memory 26 of calculating device 10. This data can be used to recalibrate BPM 12, according to a method known to one skilled in the art. Advantageously, the combination of calibration data, e.g. TP_{L} and TP_{H} and SP_{L} and SP_{H}, provides a set of data for efficient and effective calibration because, in part, the data includes controlled and sensed data at relatively low and high pressure points.

As required, detailed embodiments of the present invention are disclosed herein. However, it is to be understood that the disclosed embodiments are merely exemplary of an invention that may be embodied in various and alternative forms. Therefore, specific functional details disclosed herein are not to be interpreted as limiting, but merely as a representative basis for the claims and/or as a representative basis for teaching one skilled in the art to variously employ the present invention.

What is claimed is:

1. A method for calibrating a blood pressure monitor, the method comprising:
generating a first training pressure point signal corresponding to a first pressure value;
transmitting the first training pressure point signal to a pressure sensor of a blood pressure monitor (BPM);
receiving a first sensed pressure value sensed by the pressure sensor;
generating a second training pressure point signal corresponding to a second pressure value;
transmitting the second training pressure point signal to the pressure sensor of the BPM;
receiving a second sensed pressure value sensed by the pressure sensor;
receiving BMP calibration data; and
generating new calibration data based on the first pressure value, the second pressure value, the first sensed pressure value, the second sensed pressure value and the BMP calibration data.

2. The method of claim 1, wherein the first pressure value is a relatively low pressure value in the range of 200 to 250 mm Hg and the second pressure value is a relatively high pressure value in the range of 300 to 350 mm Hg.

3. The method of claim 1, further comprising transmitting the new calibration data to the BMP for recalibrating the BMP.

4. The method of claim 1, wherein the transmitting steps are carried out wirelessly.

5. The method of claim 1, further comprising receiving a number of uses value since the last BMP recalibration, and if the value equals a recalibration value, then executing the steps of claim 1.

6. The method of claim 5, wherein the recalibration value is in the range of 2,000 to 15,000 uses.

7. A calibration device for a blood pressure monitor comprising:
a central processing unit (CPU) for executing machine instructions;
a memory for storing machine instructions that are to be executed by the CPU;
a communication interface for electrically communicating with a blood pressure monitor, wherein the machine instructions when executed by the CPU implementing the following functions:
receiving a first training pressure point signal corresponding to a first pressure value;
transmitting the first training pressure point signal to a pressure sensor of a blood pressure monitor (BPM);
receiving a first sensed pressure value sensed by the pressure sensor;
generating a second training pressure point signal corresponding to a second pressure value;
transmitting the second training pressure point signal to the pressure sensor of the BPM;
receiving a second sensed pressure value sensed by the pressure sensor;
receiving BMP calibration data; and
generating new calibration data based on the first pressure value, the second pressure value, the first sensed pressure value, the second sensed pressure value and the BMP calibration data.

8. The calibration device of claim 7, wherein the first pressure value is a relatively low pressure value in the range of 200 to 250 mm Hg and the second pressure value is a relatively high pressure value in the range of 300 to 350 mm Hg.

9. The calibration device of claim 7, wherein the machine instructions when executed by the CPU further implement the following functions:
wirelessly transmitting the first and second training pressure point signals via the communication interface to the BPM.

10. The calibration device of claim 7, further comprising an air pressure circuit for generating the first and second training pressure point signals.

11. The calibration device of claim 10, wherein the air pressure circuit includes a serial port for connecting the air pressure circuit to a pressure sensor of a blood pressure monitor.

12. The calibration device of claim 11, wherein the air pressure circuit includes a processor for executing machine instructions to implement the following functions:
transmitting the first and second training pressure point signals to the pressure sensor of the blood pressure monitor through the serial port.

13. The calibration device of claim 7, wherein the machine instructions when executed by the CPU further implement the following function:
receiving a number of uses value since the last BMP recalibration, and if the value equals a recalibration value, then executing the steps of claim 7.

14. The calibration device of claim 13, wherein the recalibration value is in the range of 2,000 to 15,000 uses.

15. A calibration device for a blood pressure monitor comprising:
a central processing unit (CPU) for executing machine instructions;
a memory for storing machine instructions that are to be executed by the CPU;
a communication interface for electrically communicating with a blood pressure monitor; and
a pressure sensor for sensing a pressure value corresponding to a pressure point signal, wherein the machine instructions when executed by the CPU implementing the following functions:
receiving a pressure point signal (PS) corresponding to a pressure value (PV);
transmitting the PS to the pressure sensor and the communication interface;
receiving a sensed pressure value (SP_{B}) from the blood pressure monitor through the communication interface;
receiving a sensed pressure value (SP_{C}) from the pressure sensor;
comparing the difference between SP_{B} and SP_{C} with a tolerable range to obtain a calibration status of either “yes” if the difference is outside of the tolerable range and “no” if the difference is within the tolerable range; and
transmitting the calibration status to determine whether to initiate a calibration sequence.

16. The calibration device of claim 15, wherein the machine instructions when executed by the CPU further implement the following functions:
wirelessly transmitting PS via the communication interface to the blood pressure monitor.

17. The calibration device of claim 15, wherein the tolerable range is 0 to 20 mm Hg.

18. The calibration device of claim 15, wherein the tolerable range is 0 to 10 mm Hg.

19. The calibration device of claim 15, further comprising an air pressure circuit for generating PS.

20. The calibration device of claim 19, wherein the machine instructions when executed by the air pressure circuit further implement the following functions:
generating a new PS corresponding to a different pressure value than the old PS.
